(19) United States
(12) Patent Application Publication
(10) Pub. No.: US 2015/0045232 A1
(43) Pub. Date: Feb. 12, 2015

(54) INTEGRATED AND VERSATILE METHODS FOR SYSTEMS DIAGNOSIS OF DISEASES

(71) Applicant: BexMart, San Francisco, CA (US)

(72) Inventors: Bob Han, SAN FRANCISCO, CA (US); Eric Han, SAN FRANCISCO, CA (US); Xiaohui Xiong, SAN FRANCISCO, CA (US); Xiaoliang Han, SAN FRANCISCO, CA (US)

(73) Assignee: BexMart, San Francisco, CA (US)

(21) Appl. No.: 14/201,879

(22) Filed: Mar. 9, 2014

Related U.S. Application Data
(63) Continuation-in-part of application No. 13/960,548, filed on Aug. 6, 2013, now Pat. No. 8,722,337.

(51) Int. Cl. C12Q 1/68; C12N 15/10

(52) U.S. Cl. CPC C12Q 1/6806; C12N 15/1003; C12N 15/101; C12N 15/1013; C12Q 1/6886; C12Q 2600/178
USPC 506/2; 435/306.1; 435/7.1; 435/29; 435/6.11; 435/5; 435/6.12; 506/9; 435/7.92; 435/6.14; 506/39

(57) ABSTRACT

The disclosure relates to a system diagnosis of a disease or a disease condition whereby a single sample is prepared from a biological specimen which integrates synchronously the methods of protection, isolation and alteration of a biological specimen or a bio-molecule to isolate and study tissues and bio-molecules including DNA, large RNA, small RNA, protein, lipid, carbohydrates, and other metabolite simultaneously or individually resulting in a comprehensive understanding of the cause, prevention, risk, seriousness, confirmation, treatment, triage, and prognosis of a disease or a disease condition.

### CROSS-REFERENCE TO RELATED APPLICATIONS

This application claims priority from and is a cont-in-part (CIP) of U.S. non-provisional patent application Ser. No. 12/979,383 filed on Dec. 28, 2010 which are incorporated herein by reference.

### FIELD OF TECHNOLOGY

This disclosure generally relates to an integrated and versatile method to prepare a biological specimen and its comprehensive analyses. More specifically, this disclosure relates to a comprehensive and systemic diagnosis of a disease or a disease condition from an analysis of a biological specimen.

### BACKGROUND

Currently used methods for the diagnosis of a disease are individually tested for a particular bio-molecule and different bio-molecules are then combined for a diagnosis outcome. Further, a number of samples are drawn from a single specimen and are then subjected to different treatments for a single bio-molecule sample preparation and analysis. This is a very old and prevalent practice in clinical world which adds to the complexity of the diagnosis of a particular disease or a diseased condition.

As a result of this practice, the diagnosis and ultimately the treatment strategies for a particular disease are unsuccessful. There is a need for a comprehensive diagnosis method for a better management of the specimens leading to a better diagnosis and treatment.

### SUMMARY

The present application discloses a system diagnosis for a disease. Further, the present application discloses a system diagnosis for analyzing a cause, prevention, risk, seriousness, confirmation, treatment, triage and prognosis of a disease. More specifically, the present application discloses a system diagnosis by testing a specimen along with its biomolecules and metabolites from a single sample.

In one embodiment, the present application discloses an integrated and versatile method for a preparation of a biological specimen. In another embodiment, the present application discloses an integrated and a versatile method for a preparation of a clinical biological specimen. In most embodiments, the present application discloses an integrated and a versatile method for a preparation of a single sample comprising different biological metabolites of interest from a biological specimen. an analysis of a bio-molecule of interest isolated from the sample and analyzing a result for diagnosis of a disease or a disease condition. Further, in some embodiments, the results will also help in designing a treatment strategy for a disease or a disease condition.

In most embodiments, a sample prepared for the analysis of a single bio-molecule or a plurality of bio-molecules isolated from a biological specimen such as a clinical specimen. The bio-molecules of interest may comprise of DNA, mRNA, miRNA, protein, lipid, carbohydrate and other metabolites. Further, the bio-molecules may also include DNA/ccfDNA, Large RNA/mRNA/ccfRNA, Small RNA/miRNA/ccfmiRNA, metabolic proteins, signaling proteins, structural proteins, immune system proteins, membrane proteins, cellular proteins, transcription and translation protein, lipids, carbohydrates, and other metabolites.

In one embodiment, a system diagnosis of a disease comprise of preparing a sample from a biological specimen; protecting the sample; isolating a bio-molecule from a sample; estimating the bio-molecule; and analyzing the result for the diagnosis of a disease or a disease condition. In another embodiment, a system diagnosis of a disease comprise of preparing a sample from a biological specimen; protecting the sample; isolating a plurality of bio-molecules from a sample; estimating the bio-molecules; and analyzing the results for the diagnosis of a disease or a disease condition. In most embodiments, results of the assays performed such as (but not limited to) a molecular test, an immunoassay, a cytology study, a histology study will lead to a better diagnosis (or a pre-diagnosis) of a disease or a disease condition, helping in better prognosis and thus a treatment triage and a treatment strategy.

In one embodiment, a system diagnosis comprise of contacting a protecting, lysis and inhibiting solution to a biological specimen; forming a homogenate or a lysate; subjecting the homegenate/lysate to a differential centrifugation followed by a differential precipitation to form a sample; isolating a bio-molecule from a sample; assessing the bio-molecule; and analyzing the result for the diagnosis of a disease or a disease condition. In another embodiment, a system diagnosis comprise of contacting a protecting, lysing and inhibiting solution to a biological specimen; forming a homogenate/a lysate; subjecting the homogenate/lysate to a differential centrifugation followed by a differential precipitation to form a sample; isolating a plurality of bio-molecules from the sample; performing an assay to estimate isolated bio-molecules; analyzing the assay results for the diagnosis of a disease or a disease condition. In some embodiments, a system diagnosis comprise of contacting a protecting, lysing and inhibiting solution to a clinical biological specimen; forming a homogenate/lysate; subjecting the homogenate/lysate to a differential centrifugation followed by a differential precipitation to form a sample; isolating DNA, mRNA, miRNA, protein, lipid, carbohydrate from the sample; performing an assay to estimate isolated DNA, mRNA, miRNA, protein, lipid, carbohydrate moieties; analyzing the assay results for the diagnosis of a disease or a disease condition.

In one embodiment, a system diagnosis comprise of contacting a protecting, lysis and inhibiting solution to a biological specimen; forming a homogenate or a lysate; subjecting the homegenate/lysate to a differential solid phase binding followed by a differential precipitation to form a sample; isolating a bio-molecule from a sample; assessing the bio-molecule; and analyzing the result for the diagnosis of a disease or a disease condition. In another embodiment, a system diagnosis comprise of contacting a protecting, lysing and inhibiting solution to a biological specimen; forming a homogenate/a lysate; subjecting the homogenate/lysate to a differential solid phase binding Followed by a differential precipitation to form a sample; isolating a plurality of bio-molecules from the sample; performing an assay to estimate isolated bio-molecules; analyzing the assay results for the diagnosis of a disease or a disease condition. In some embodiments, a system diagnosis comprise of contacting a protecting, lysing and inhibiting solution to a clinical biological specimen; forming a homogenate/lysate; subjecting the homogenate/lysate to a differential solid phase binding followed by a differential precipitation to form a sample; isolating DNA, mRNA, miRNA, protein, lipid, carbohydrate from the sample; performing an assay to estimate isolated DNA, mRNA, miRNA, protein, lipid, carbohydrate moieties; analyzing the assay results for the diagnosis of a disease or a disease condition.

In one embodiment, a system diagnosis comprise of contacting a protecting, lysis and inhibiting solution to a biological specimen; forming a homogenate or a lysate; subjecting the homegenate/lysate to a differential solid phase binding or a differential centrifugation followed by a differential precipitation to form a sample; isolating a bio-molecule from a sample; assessing the bio-molecule; and analyzing the result for the diagnosis of a disease or a disease condition. In another embodiment, a system diagnosis comprise of contacting a protecting, lysing and inhibiting solution to a biological specimen; forming a homogenate/a lysate; subjecting the homogenate/lysate to a differential solid phase binding or a differential centrifugation followed by a differential precipitation to form a sample; isolating a plurality of bio-molecules from the sample; performing an assay to estimate isolated bio-molecules; analyzing the assay results for the diagnosis of a disease or a disease condition. In some embodiments, a system diagnosis comprise of contacting a protecting, lysing and inhibiting solution to a clinical biological specimen; forming a homogenate/lysate; subjecting the homogenate/lysate to a differential solid phase binding or a differential centrifugation followed by a differential precipitation to form a sample; isolating DNA, mRNA, miRNA, protein, lipid, carbohydrate from the sample; performing an assay to estimate isolated DNA, mRNA, miRNA, protein, lipid, carbohydrate moieties; analyzing the assay results for the diagnosis of a disease or a disease condition.

In one embodiment, a single sample is prepared for a biological specimen for the diagnosis of a disease or a disease condition whereas in another embodiment, a single sample is prepared from a clinical biological specimen for the diagnosis of a disease or a disease condition. Thus, the present disclosure relates to an isolation of plurality of bio-molecules from a single sample prepared from a biological specimen leading to a systemic and better diagnosis.

In most embodiments, a clinical specimen as disclosed may be a solid specimen or a liquid specimen. Further, the specimen may also be a clinical specimen, a non-clinical specimen, a clinical biopsy tissue, surgical tissue, buccal cells, smear of body fluids containing cells or tissues on glass slides or other supporting materials, swab contacting body fluids containing cells or tissues, cells or tissue from fine needle aspiration of mass, and cells or tissue from endobronchial ultrasound with transbronchial needle aspiration, microorganisms, whole blood, plasma, serum, buffy coat, bone marrow, amniotic fluid, bronchial aspirates, cerebrospinal fluid, cervical and vaginal secretions, mucus, peritoneal fluid, ascite, pleural effusion, saliva, semen, sputum, synovial fluid, and urine. Further, a clinical specimen may be a specimen from (but not limited to) a cervical cancer patient, a lung cancer patient, a prostate cancer patient, a colorectal cancer patient, a melanoma patient, a lymphoma patient, and a thyroid cancer patient.

In most embodiments, the bio-molecules as disclosed may comprise of (but not limited to) genomic DNA (gDNA), circulating cell free DNA (ccfDNA), circulating cell free genomic DNA (ccfgDNA), nucleic DNA (nDNA), mitochondria DNA (mitDNA), chloroplast DNA (cpDNA), plasmid DNA (pDNA), virus DNA (vDNA), complementary DNA (cDNA), converted DNA (cvDNA), messenger ribonucleic acid (mRNA), circulating cell free RNA (ccfRNA), circulating cell free mRNA (ccfmRNA), heterogeneous nuclear RNA (hnRNA), ribosomal ribonucleic acid (rRNA), transfer-messenger RNA (tmRNA), antisense RNA (aRNA), virus RNA (vRNA), microRNA (miRNA), transfer RNA (tRNA), circulating cell free small RNA (ccfsRNA), circulating cell free microRNA (ccfmiRNA), small interfering RNA (siRNA), piwi-interacting RNA (piRNA), small nuclear ribonucleic acid (snRNA), small nucleolar RNAs (snoRNAs), trans-acting siRNA (ta-siRNA/tasiRNA), guide RNAs (gRNA), small temporal RNA (stRNA), small hairpin RNA/short hairpin RNA (shRNA), total protein, cytoplasmic protein, nucleic protein, membrane protein, cytoskeleton protein, transcription and translation protein, membrane protein, total lipids, membrane lipids and other bio-molecules and metabolites. Further, a system diagnosis may also comprise of performing morphological analysis, gene alteration studies and pathogen tests. Thus, results from different bio-molecules assessments, morphological studies and other assays help in diagnosis and treatment of a disease or a disease condition.

In one embodiment, system diagnosis comprise of collecting a specimen; contacting a protecting to the specimen; preparing the specimen for a morphological test; performing the morphological test and analyzing results for diagnosis of a disease or a disease condition. In another embodiment, system diagnosis comprise of: collecting a specimen; contacting a protecting to the specimen; preparing the specimen for a morphological study; wherein the morphological study may include a cytology test, a histology test, a fluorescence activated cell sorting (FACS) study, an immunocytochemistry (ICC) study; an immunohistochemistry (IHC) study, in situ hybridization (ISH), performing the morphological study and analyzing analyzing results for diagnosis of a disease or a disease condition.

In one embodiment, a system diagnosis comprise of contacting a protecting, lysis and inhibiting solution to a biological specimen; forming a homogenate/lysate; subjecting the homegenate/lysate to a differential centrifugation followed by a differential precipitation to form a sample; isolating DNA/ccDNA from the sample; performing a mutation study; and analyzing the result for the diagnosis of a disease or a disease condition. In another embodiment, a system diagnosis comprise of contacting a protecting, lysis and inhibiting solution to a biological specimen; forming a homogenate or a lysate; subjecting the homegenate/lysate to a differential centrifugation followed by a differential precipitation to form a sample; isolating large RNA/mRNA/ccfRNA/small RNA/miRNA/ccfRNA from a sample; performing a expression study; and analyzing the result for the diagnosis of a disease or a disease condition. In some embodiment, a system diagnosis comprise of contacting a protecting, lysis and inhibiting solution to a biological specimen; forming a homogenate or a lysate; subjecting the homegenate/lysate to a differential centrifugation followed by a differential precipitation to form a sample; isolating plurality of proteins from a sample; performing a protein expression study; and analyzing the result for the diagnosis of a disease or a disease condition.

In one embodiment, a system diagnosis comprise of contacting a protecting, lysis and inhibiting solution to a biological specimen; forming a homogenate/lysate; subjecting the homegenate/lysate to a differential solid phase binding followed by a differential precipitation to form a sample; isolating DNA/ccDNA from the sample; performing a mutation study; and analyzing the result for the diagnosis of a disease or a disease condition. In another embodiment, a system diagnosis comprise of contacting a protecting, lysis and inhibiting solution to a biological specimen; forming a homogenate or a lysate; subjecting the homegenate/lysate to a differential centrifugation followed by a differential precipitation to form a sample; isolating large RNA/mRNA/ccfRNA/small RNA/miRNA/ccfRNA from a sample; performing a expression study; and analyzing the result for the diagnosis of a disease or a disease condition. In some embodiment, a system diagnosis comprise of contacting a protecting, lysis and inhibiting solution to a biological specimen; forming a homogenate or a lysate; subjecting the homegenate/lysate to a differential centrifugation followed by a differential precipitation to form a sample; isolating plurality of proteins from a sample; performing a protein expression study; and analyzing the result for the diagnosis of a disease or a disease condition.

In one embodiment, a system diagnosis comprise of contacting a protecting, lysis and inhibiting solution to a biological specimen; forming a homogenate/lysate; subjecting the homegenate/lysate to a differential solid phase binding or a differential centrifugation followed by a differential precipitation to form a sample; isolating DNA/ccDNA from the sample; performing a mutation study; and analyzing the result for the diagnosis of a disease or a disease condition. In another embodiment, a system diagnosis comprise of contacting a protecting, lysis and inhibiting solution to a biological specimen; forming a homogenate or a lysate; subjecting the homegenate/lysate to a differential solid phase binding or a differential centrifugation followed by a differential precipitation to form a sample; isolating large RNA/mRNA/ccfRNA/small RNA/miRNA/ccfRNA from a sample; performing a expression study; and analyzing the result for the diagnosis of a disease or a disease condition. In some embodiment, a system diagnosis comprise of contacting a protecting, lysis and inhibiting solution to a biological specimen; forming a homogenate or a lysate; subjecting the homegenate/lysate to a differential solid phase binding or a differential centrifugation followed by a differential precipitation to form a sample; isolating plurality of proteins from a sample; performing a protein expression study; and analyzing the result for the diagnosis of a disease or a disease condition.

In one embodiment, system diagnosis comprise of collecting a clinical biological specimen; wherein the clinical biological specimen is from a cancer patient, contacting a protecting, lysis and inhibiting solution to the specimen; forming a homogenate or a lysate; subjecting the homegenate/lysate to a differential differential centrifugation followed by a differential precipitation to form a sample; isolating a bio-molecule from a sample; estimating the bio-molecule; and analyzing the result for the diagnosis of cancer. In another embodiment, a system diagnosis comprise of collecting a clinical biological sample; wherein the clinical biological sample is a cervical cancer tissue, contacting a protecting, lysing and inhibiting solution to a biological sample; forming a homogenate or a lysate; subjecting the homogenate or a lysate to a differential centrifugation followed by a differential precipitation to form a sample; isolating plurality of bio-molecules from the sample; performing the assay to estimate isolated bio-molecules; analyzing the assay results for the diagnosis of cervical cancer.

In one embodiment, system diagnosis comprise of collecting a clinical biological specimen; wherein the clinical biological specimen is from a cancer patient, contacting a protecting, lysis and inhibiting solution to the specimen; forming a homogenate or a lysate; subjecting the homegenate/lysate to a differential solid phase binding followed by a differential precipitation to form a sample; isolating a bio-molecule from a sample; estimating the bio-molecule; and analyzing the result for the diagnosis of cancer. In another embodiment, a system diagnosis comprise of collecting a clinical biological sample; wherein the clinical biological sample is a cervical cancer tissue, contacting a protecting, lysing and inhibiting solution to a biological sample; forming a homogenate or a lysate; subjecting the homogenate or a lysate to a differential solid phase binding followed by a differential precipitation to form a sample; isolating plurality of bio-molecules from the sample; performing the assay to estimate isolated bio-molecules; analyzing the assay results for the diagnosis of cervical cancer.

In one embodiment, system diagnosis comprise of collecting a clinical biological specimen; wherein the clinical biological specimen is from a cancer patient, contacting a protecting, lysis and inhibiting solution to the specimen; forming a homogenate or a lysate; subjecting the homegenate/lysate to a differential solid phase binding or a differential centrifugation followed by a differential precipitation to form a sample; isolating a bio-molecule from a sample; estimating the bio-molecule; and analyzing the result for the diagnosis of cancer. In another embodiment, a system diagnosis comprise of collecting a clinical biological sample; wherein the clinical biological sample is a cervical cancer tissue, contacting a protecting, lysing and inhibiting solution to a biological sample; forming a homogenate or a lysate; subjecting the homogenate or a lysate to a differential solid phase binding or a differential centrifugation followed by a differential precipitation to form a sample; isolating plurality of bio-molecules from the sample; performing the assay to estimate isolated bio-molecules; analyzing the assay results for the diagnosis of cervical cancer.

The systems diagnosis of cervical cancer is achieved by integrating comprehensive parameters from preventive screen test of high risk Human PapillomaVirus (HPV) DNA to identify high risk of population to who may suffer from cervical cancer in the future. Further, in many embodiments, diagnosis of cervical cancer may also be done by performing a test of E6/E7 mRNA and a E6/E7 protein for a treatment triage; a test of a micromorphology change of an epithelia cell to screen a population who may suffer from cervical pre-cancer condition; a test of any histology change to confirm the diagnosis of cervical cancer; and a test of expression signature of a miR-200a and miR-9 for cervical cancer prognosis.

In one embodiment, the present disclosure also relates to a kit, comprising: a core module, a decontaminant solution, a lysis solution, a plug-in module for the isolation of a bio-molecule wherein the bio-molecule is analyzed to diagnosis a disease or a disease condition. The kit as disclosed further comprising: one or more plug-in modules for isolation of a number of bio-molecules from a biological specimen wherein the bio-molecule is analyzed to diagnosis a disease or a disease condition. In some embodiments, the kit may further comprise a staining kit to carry out morphological study wherein structural studies are done to diagnose a disease or a disease condition.

Thus, the present disclosure relates to a versatile and integrated method to prepare a single sample from a single specimen wherein the specimen may be a clinical specimen such as a cervical cancer specimen; and using the sample to analyze a number of bio-molecules and biomarkers for the screening or pre-screening of a disease or a disease condition followed by developing a treatment triage.

Other features will be apparent from the accompanying figures and from the detailed description that follows.

### BRIEF DESCRIPTION OF THE DRAWINGS

Example embodiments are illustrated by way of example and no limitation in the accompanying figures and tables, like references indicate similar elements and in which:

FIG. 1 shows an overview of an integrated and versatile method for systems diagnosis of diseases.

FIG. 2 shows a protection of histological structure and micromorphology of specimens by the treatment solutions.

FIG. 3 shows an integrated and versatile methods for systems preparation of a clinical biological specimen.

FIG. 4 shows an integrated and versatile method for systems diagnosis of cervical cancer.

### DETAILED DESCRIPTION

The present disclosure relates to an integrated and versatile method for a sample preparation and analyzing the sample for diagnosis and treatment of a disease or a disease condition.

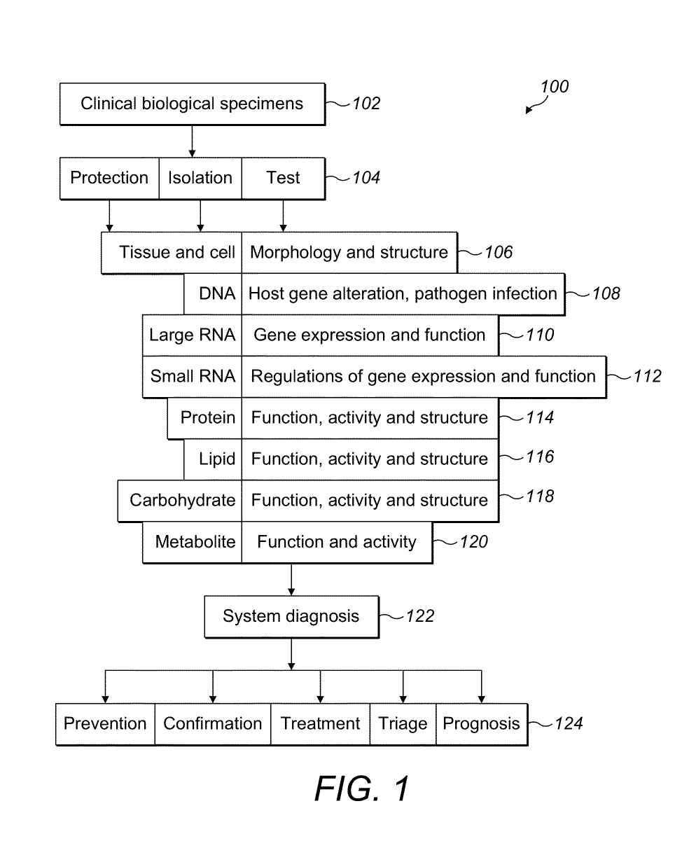

FIG. 1 gives an overview of an integrated and a versatile method for systems diagnosis of a disease or a disease condition. As can be seen from FIG. 1, a clinical biological specimen 102 is subjected to protection and isolation 104 followed by the lysis of the specimen forming a lysate. The lysate is then subjected to a differential solid phase binding or a differential centrifugation and different precipitation subjecting to various inhibiting solutions leading to the isolation of different bio-molecules such as DNA 108/large RNA 110/small RNA 112/protein 114/lipid 116/carbohydrate 118 and other metabolite 120. Further, the bio-molecules as separated will then be analyzed under different tests which may include (but not limited to) host gene alteration studies 108, pathogen infection studies 108, gene expression and functional studies 110, regulation studies 112, function, activity and structural studies 114-120 respectively. The results from all the above mentioned tests are then analyzed through system diagnosis 122 to study the prevention, confirmation, treatment, triage and prognosis 124 of a disease or a disease condition. The clinical biological specimen as specified may be a tissue or a cell(s).

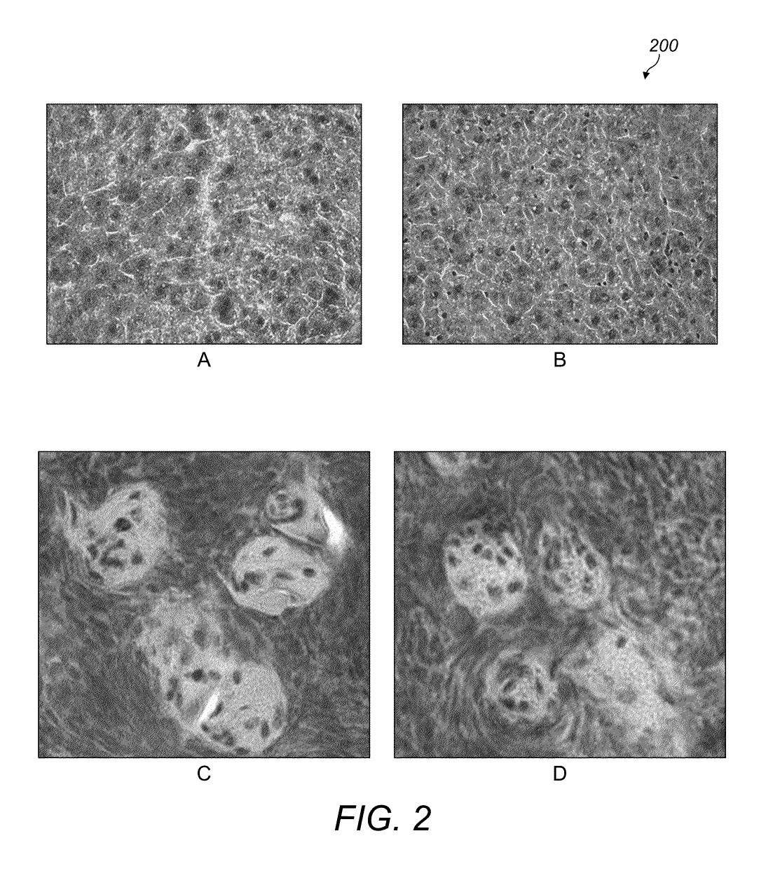

Protection of the specimen, protects and preserve structure and composition of specimen (FIG. 2) which may be analyzed through morphological and structural studies 106 on the protected specimen as also shown in FIG. 1, which include but not limited to cytology, histology, FACS, ICC, IHC, ISH and FISH. FIG. 2 shows results of using the protection solution onto the clinical biological specimen. Panel A shows a frozen liver tissue section with hemotoxylin-eosin (HE) staining after storing in a treatment solution at 4° C. for 10 days; Panel B shows a frozen liver tissue section with HE staining after store in −80° C. freezer for 10 days without treatment solution; Panel C shows a FFPE human lung cancer small cell carcinoma tissue section with HE staining after store in treatment solution at 4° C. for 10 days; and Panel D shows a FFPE human lung cancer small cell carcinoma tissue section with HE staining after store in −80° C. freezer for 10 days without treatment solution. Treatment solution protects the histological structure and micromorphology of specimens when specimens were stored in treatment solution at 4° C. for 10 days. Stored specimens can be used for frozen or FFPE section.

There are two common terms to describe or definite a piece or a volume of object taken from an organism in biomedical field, a specimen or a sample. Both the terms are interchangeable in many places throughout the specification. Although the term of the specimen is selected in the invention due to its precision and broadness to the object, the term of the sample is still applicable in the places where specimen is used in this invention. The clinical biological specimen as in FIG. 1 may be a solid or a liquid specimen including, (but not limited to) clinical sample, non-clinical sample, clinical biopsy tissue, surgical tissues, buccal cells, smear of body fluids containing cells or tissues on glass slides or other supporting materials, swab contacting body fluids containing cells or tissues, cells or tissue from fine needle aspiration of mass, and cells or tissue from endobronchial ultrasound with transbronchial needle aspiration, microorganisms, whole blood, plasma, serum, buffy coat, bone marrow, amniotic fluid, bronchial aspirates, cerebrospinal fluid, cervical and vaginal secretions, mucus, peritoneal fluid, ascite, pleural effusion, saliva, semen, sputum, synovial fluid, and urine (Table 1).

TABLE 1
List of clinical biological specimen.

| Solid Specimens | Liquid Specimens | Other BioFluid |
|---|---|---|
| Tissues | Plasma | Cerebrospinal fluid |
| Frozen tissue sections | Serum | Gastric juice |
| FFPE tissue sections | Whole blood | Pleural effusion |
| Biopsy samples | Buffy coat | Ascite |
| Cell pellet | Bone marrow | Synovial fluid |
| Attached cells | Cell suspension | Tears |
| Buccal cells | Saliva | Semen |
| Microorganisms | Urine | Sweat |
| Bacteria | Biofluids | Sputum |
| Yeast | Beverage | Cervical and |
| Virus | Water | vaginal secretions |
| Parasite | RNA contaminated with | Feces, stool, |
| Stool | DNA | excrement |
| Soil | PCR reaction | Mucus |
| Transport media | Enzymatic reaction | |
| Blood spots | Sequencing reaction | |
| Blood cards | Labeling reaction | |
| Swabs | Chemical modified samples | |
| Plants | Contaminated samples | |
| Gel fraction | Diluted samples | |
| Food | | |

Further, the bio-molecules as disclosed (FIG. 1) above may be (but not limited to) DNA/ccfDNA, Large RNA/mRNA/ccfRNA, Small RNA/miRNA/ccfmiRNA, protein, lipid, carbohydrate, and metabolite as listed in Table 2.

TABLE 2
List of bio-molecules derived from a clinical biological specimen.

| DNA/ccfDNA | Large RNA/mRNA/ccfRNA |
|---|---|
| Genomic DNA (gDNA) | Messenger ribonucleic acid (mRNA) |
| Circulating cell free DNA (ccfDNA) | Circulating cell free RNA (ccfRNA) |
| Circulating cell free genomic DNA (ccfgDNA) | Circulating cell free mRNA (ccfmRNA) |
| Nucleic DNA (nDNA) | Heterogeneous nuclear RNA (hnRNA) |
| Mitochondria DNA (mitDNA) | Ribosomal ribonucleic acid (rRNA) |
| Chloroplast DNA (cpDNA) | Transfer-messenger RNA (abbreviated |
| Plasmid DNA (pDNA) | tmRNA) |
| Virus DNA (vDNA) | Antisense RNA (aRNA) |
| Complementary DNA (cDNA) | Virus RNA (vRNA) |
| Converted DNA (cvDNA) | |
| Small RNA/miRNA/ccfmiRNA (less than 100 bp) | Protein |
| MicroRNA (miRNA) | Total protein |
| Transfer RNA (tRNA) | Cytoplasmic protein |
| Circulating cell free small RNA (ccfsRNA) | Nucleic protein |
| Circulating cell free microRNA (ccfmiRNA) | Membrane protein |
| Small interfering RNA (siRNA) | Cytoskeleton protein |
| Piwi-interacting RNA (piRNA) | |
| Small nuclear ribonucleic acid (snRNA) | |
| Small nucleolar RNAs (snoRNAs) | |
| Trans-acting siRNA (ta-siRNA or tasiRNA) | |
| Guide RNAs (aka gRNA) | |
| Small temporal RNA (abbreviated stRNA) | |
| Small hairpin RNA or short hairpin RNA (shRNA) | |
| Lipid | Carbohydrate |
| Metabolite | Others |

Thus, the system diagnosis comprise of a protected specimen and isolated bio-molecules which are then subjected to various morphological, biochemical, biological and other known assays.

Further, the present disclosure also relates to study alterations that may be introduced into the specimen (clinical biological specimen) before it is subjected to isolation and preparation steps. The protected specimen and the isolated bio-molecules (after the introduction of an alteration) are then subjected to various known and used assays to see the result of alterations onto the specimen. The results lead to a comprehensive diagnosis of a disease or a disease condition which leads to a better prognosis and help in designing treatment strategies.

Further, the present application uses a single sample isolated from a single clinical biological sample for the comprehensive analysis and helping medical practitioners to develop treatment strategies.

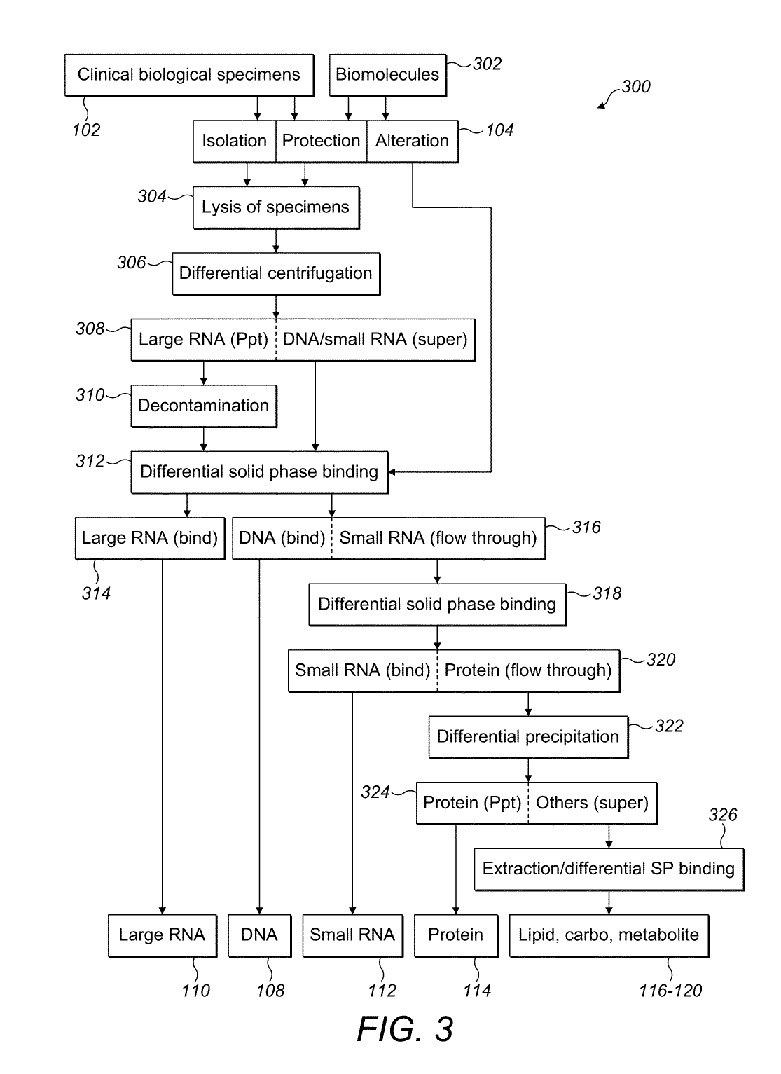

FIG. 3 shows an integrated and a versatile method for systems preparation of a clinical biological specimen. As shown in FIG. 3, clinical biological specimens 102 are subjected to isolation and protection 104. However, if one is using bio-molecules 302, they can be subjected to alterations 104 and can be protected 104 for morphological studies. The specimen is subjected to lysis 304 forming a lysate which is then introduced to a differential centrifugation 306 resulting in isolation of a large RNA in precipitate 308 and DNA and small RNA in the supernatant 308. The precipitate is then decontaminated 310 followed by differential solid phase binding 312 leading to the separation of a large RNA (binds to the solid phase) 314, DNA (binds to the solid phase) and a small RNA (that flows through) 316. The supernatant 308 is also subjected to differential solid phase binding (without subjecting to decontamination) and leads to the separation of DNA (binds to a solid phase) and small RNA (flows through) 316. Small RNA 316 is further subjected to a differential solid phase binding 318 leading to the separation of a small RNA (binding to the solid phase 320) and proteins which flows through 320. The proteins are subjected to a differential precipitation 322 leading to the separation of proteins (in precipitate 324) from other bio-molecules in supernatant 324. The supernatant with other bio-molecules is subjected to an extraction/differential solid phase binding 326 for the isolation of lipids, carbohydrates and other metabolites 116-120. Thus, we will have isolated large RNA 110, DNA 108, small RNA 112, protein 114, lipid, carbohydrate and other metabolites 116-120 from a single specimen leading to a much better and reliable diagnosis of a disease or a disease condition which will eventually affect the treatment strategy.

One may also introduce desired alterations in the bio-molecules to study its affect in disease condition. In this case, the bio-molecules are subjected directly to a differential solid phase binding 312 and further process for the isolation of different bio-molecules and metabolites as described above.

The differential centrifugation process and a differential precipitation process on the homogenate/lysate to separate the large RNA/mRNA/ccfRNA of the lysate comprises the steps of: (a) centrifuging the homogenate or lysate to separate and collect the large RNA/mRNA/ccfRNA as pellet 1 away from a supernatant (1) comprising DNA/ccfDNA, small RNA/miRNA/ccfmiRNA and protein; (b) dissolving the pellet 1 comprising the large RNA/mRNA/ccfRNA in a decontamination solution comprising sodium iodide at 1-5 M, ammonium acetate at 1-10 M, sodium acetate at 0.1-5M, sodium citrate at 10-100 mM, sodium chloride (NaCl) at 0.1-5 M, and ethylene diamine tetraacetic acid disodium salt dihydrate (EDTA) at 0.1-50 mM; (c) periodically vortex the pellet 1 to dissolve the pellet thoroughly in the decontamination solution for five seconds at a time; (d) centrifuging the dissolved pellet 1 of step (c) at 13000 rpm for 2 min at room temperature to remove insoluble contaminants as a new pellet and recover the large RNA/mRNA/ccfRNA in a supernatant (2); (e) adding ethanol to the supernatant 2 to form a mixture comprising precipitated large RNA/mRNA/ccfRNA; (f) centrifuging the mixture of step (e) to collect the precipitated large RNA/mRNA/ccfRNA as a pellet 2 and a supernatant 3 for discarding; (g) washing the pellet 2 with a 75% ethanol solution; (h) centrifuging to recollect pellet 2 as a pure sample of the large RNA/mRNA/ccfRNA after discard the supernatant 4; (i) air drying of pellet 2; and (j) dissolving the air dried pellet 2 in RNAse and DNase free water. Extracting DNA/ccfDNA from the supernatant 1 by a differential centrifugation and a differential precipitation process without toxic phenol/chloroform extraction, wherein the extracting of DNA/ccfDNA comprises: (a) adding ethanol to the supernatant 1 to precipitate the DNA/ccfDNA; (b) collecting the precipitated DNA/ccfDNA by centrifugation at 13000 rpm as a pellet 3 comprising the DNA/ccfDNA and a supernatant 5 comprising small RNA/miRNA/ccfmiRNA and protein; (c) washing the pellet 3 with a 75% ethanol solution; (d) centrifuging and collecting the pure sample of the DNA/ccfDNA.

Performing a differential solid phase binding process on the homogenate/lysate to separate the bio-molecule comprises the steps of: (a) centrifuging the homogenate/lysate to separate and collect the large RNA/mRNA/ccfRNA as pellet 1 away from a supernatant 1 comprising DNA/ccfDNA, small RNA/miRNA/ccfmiRNA and protein; (b) dissolving the pellet 1 comprising the large RNA/mRNA/ccfRNA in a decontamination solution comprising sodium iodide at 1-5 M, ammonium acetate at 1-10 M, sodium acetate at 0.1-5M, sodium citrate at 10-100 mM, sodium chloride (NaCl) at 0.1-5 M, and ethylene diamine tetraacetic acid disodium salt dihydrate (EDTA) at 0.1-50 mM; (c) periodically vortex the pellet 1 to dissolve the pellet thoroughly in the decontamination solution for five seconds at a time; (d) centrifuging the dissolved pellet 1 of step (c) at 13000 rpm for 2 min at room temperature to remove insoluble contaminants as a new pellet and recover the large RNA/mRNA/ccfRNA in a supernatant 2; (e) adding ethanol and solid phase binding materials to the supernatant 2 to bind large RNA/mRNA/ccfRNA on solid phase binding materials to form bond solid phase binding materials 1; (f) Separating the bond solid phase binding materials of step (e) to collect the bond large RNA/mRNA/ccfRNA on solid phase binding materials as the bond large RNA/mRNA/ccfRNA and a supernatant 3 for discarding; (g) washing the bond large RNA/mRNA/ccfRNA with a 75% ethanol solution; (h) Separating to recollect the bond large RNA/mRNA/ccfRNA as a pure sample of the large RNA/mRNA/ccfRNA after discard the supernatant 4; (i) air drying of the bond large RNA/mRNA/ccfRNA; and (j) eluting the air dried bond large RNA/mRNA/ccfRNA in RNAse and DNase free water. Extracting DNA/ccfDNA, small RNA/miRNA/ccfmiRNA, and protein from the supernatant 1 by a differential solid phase binding process without toxic phenol/chloroform extraction, wherein the extracting of DNA/ccfDNA, small RNA/miRNA/ccfmiRNA, and protein comprises: (a) adding ethanol and solid phase binding materials to the supernatant 1 to bind the DNA/ccfDNA to form bond solid phase binding materials 2; (b) Separating the bond solid phase binding materials 2 of step (a) to collect the bond DNA/ccfDNA on solid phase binding materials 2 as the bond DNA/ccfDNA and a supernatant 5 comprising small RNA/miRNA/ccfmiRNA and protein; (c) washing the bond DNA/ccfDNA with a 75% ethanol solution; and (d) eluting the air dried bond DNA/ccfDNA and collecting the pure sample of the DNA/ccfDNA. (e) adding ethanol and solid phase binding materials to the supernatant 5 to bind the small RNA/miRNA/ccfmiRNA to form bond solid phase binding materials 3; (f) Separating the bond solid phase binding materials 3 of step (e) to collect the bond small RNA/miRNA/ccfmiRNA on solid phase binding materials 3 as the bond small RNA/miRNA/ccfmiRNA and a supernatant 6 comprising protein; (g) washing the small RNA/miRNA/ccfmiRNA with a 75% ethanol solution; and (h) eluting the air dried bond small RNA/miRNA/ccfmiRNA and collecting the pure sample of the small RNA/miRNA/ccfmiRNA. (i) adding ethanol to the supernatant 6 to form a mixture comprising precipitated protein; (j) centrifuging the mixture of step (i) to collect the precipitated protein as a pellet 3 and a supernatant 7 containing lipid, carbohydrates, and other metabolites; (k) washing the pellet 3 with a 75% ethanol solution; (1) centrifuging to recollect pellet 3 as a pure sample of the protein after collecting the supernatant 8 for further extraction of lipid, carbohydrates, and other metabolites; (m) air drying of pellet 3; and (n) dissolving the air dried pellet 3 as protein.

The solid phase binding materials include magnetic particles coated with materials which bind nucleic acids. A number of commercially and non-commercially available maerial can be used such as a glass fiber in the format of a filter membrane or a particle (s) which bind nucleic acid(s) or any other solid phase materials coated with materials which bind nucleic acids.

The DNA/ccfDNA isolated from clinical biological specimens that may include cancer cells and normal cells and tissues includes host genomic DNA and pathogen DNA that is applied for systems diagnosis through molecular tests of mutation and quantity including, but not limited to, Polymerase Chain Reaction (PCR), hybridization, microarray, and sequencing for different cancer and diseases including, but not limited to, cervical cancer, lung cancer, prostate cancer, colorectal cancer, melanoma, lymphoma, and thyroid cancer.

The Large RNA/mRNA/ccfRNA isolated from clinical biological specimens including cancer and normal cells and tissues includes host RNA and pathogen RNA that is applied for systems diagnosis through molecular tests of expression amount and splicing including, but not limited to, Reverse Transcriptional PCR (RT-PCR), hybridization, microarray, and sequencing for different cancer and diseases including, but not limited to, cervical cancer, lung cancer, prostate cancer, colorectal cancer, melanoma, lymphoma, and thyroid cancer.

The Small RNA/miRNA/ccfmiRNA isolated from clinical biological specimens including cancer and normal cells and tissues is applied for systems diagnosis through molecular tests of expression amount including, but not limited to, RT-PCR, hybridization, microarray, and sequencing for different cancer and diseases including, but not limited to, cervical cancer, lung cancer, prostate cancer, colorectal cancer, melanoma, lymphoma, and thyroid cancer.

The Protein isolated from clinical biological specimens including cancer and normal cells and tissues is applied for systems diagnosis through different tests of protein expression amount including, but not limited to, immunoassay, immunohistochemistry, immunocytochemistry, The enzyme-linked immunosorbent assay (ELISA), mass spectrum for different cancer and diseases including, but not limited to, cervical cancer, lung cancer, prostate cancer, colorectal cancer, melanoma, lymphoma, and thyroid cancer.

The lipid, carbohydrate, and metabolite isolated from clinical biological specimens including cancer and normal cells and tissues is applied for systems diagnosis through a variety of tests for different cancer and diseases including, but not limited to, cervical cancer, lung cancer, prostate cancer, colorectal cancer, melanoma, lymphoma, and thyroid cancer.

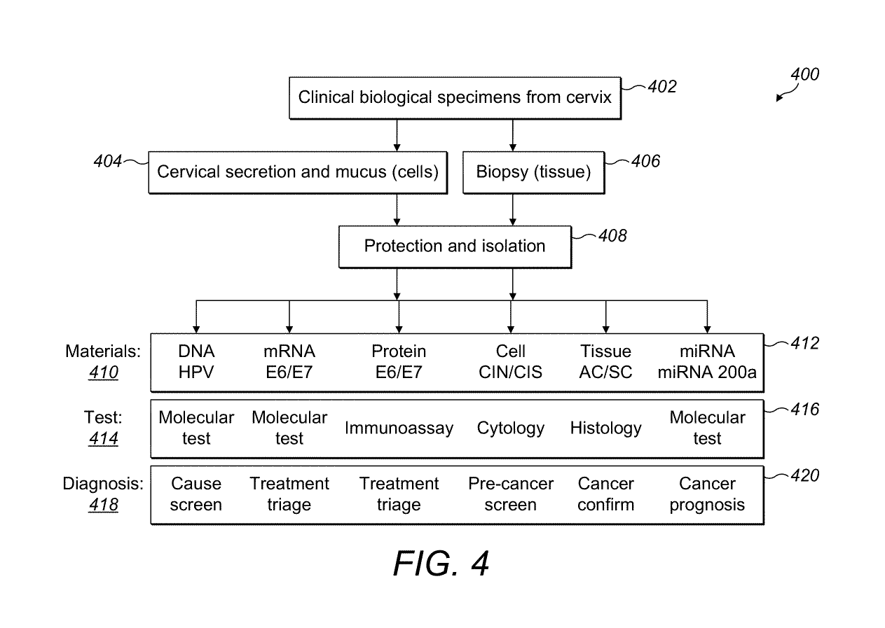

The integrated and versatile methods for systems diagnosis of disease has been implemented for cervical cancer through preparation and tests of cell, tissue and bio-molecules derived from cervical secretion and mucus containing cells (FIG. 4). Since specimens, bio-molecules, test methods and mechanism of diseases are varied among different diseases, results and contents of the systems diagnosis of diseases are slightly different from general principle as shown in FIG. 1. Human Papilloma Virus (HPV) is the cause for cervical cancer. High risk HPV, such as HPV 16 or 18 have higher potent to cause cervical cancer. After infection of HPV, patients may develop cervical cancer in a few years. World Health Organization (WHO) Histologic terminology defines the pathologic changes cause by HPV including Condyloma, Cervical Intraepithelial Neoplasia (CIN), Adenocarcinoma (AC) in Situ, micro-invasive squamous carcinoma (SC), invasive squamous or glandular carcinoma. CIN1 is mild dysplasia, Carcinoma In Situ (CIS)/CIN2/CIN3/are moderate and severe dysplasia.

As shown in FIG. 4, the materials for systems diagnosis of cervical cancer are derived from clinical biological specimens from cervix 402, which may include cells (cervical secretion and mucus 404) and tissue (biopsy 406). The cervical secretion and mucus containing cells 404 and/or tissue is treated with a protecting, lysis and inhibiting solution 408 selected from the group consisting of a variety of treatment solutions to protect histological structures and micromorphology of cells and tissues, and intactness of bio-molecules of the cervical secretion and mucus containing cells and/or to preserve the intactness of the bio-molecules in a lysate of the cervical secretion and mucus containing cells.

The protecting, lysis and inhibiting solution inhibits endogenous degrading enzymes in the cervical secretion and mucus. After lysis or homogenization of cervical secretion and mucus, HPV DNA, E6E7 mRNA, miR-200a and miR-9 miRNA, and E6E7 protein 410-412 are isolated by performing a differential centrifugation process, a differential precipitation process and a differential solid phase binding process on the lysate without toxic phenol/chloroform extraction to separate the HPV DNA, E6/E7, CIN/CIS, mRNA, miR-200a and miR-9 miRNA, and E6E7 protein.

The systems diagnosis of cervical cancer is carried out through tests of cells, tissues, and biomolecules derived from cervical secretion and mucus on aspects of cytology and histological structures and micromorphology of epithelia cells and biopsy tissues 414-416; molecular biology test of HPV DNA, E6/E7 mRNA, miR-200a and miR-9; and immunology test of E6/E7 protein. The systems diagnosis of cervical cancer is achieved by integrating comprehensive parameters from preventive screen test of high risk HPV DNA to identify high risk of population to suffer cervical cancer in the future; test of micromorphology changes of epithelia cells to screen population to have cervical cancer; test of E6/E7 mRNA and E6/E7 protein for treatment triage, and test of expression signature miR-200a and miR-9 for cervical cancer prognosis.

The analysis of these test results leads to a system diagnosis and helps in screening a cause, a treatment triage, pre-cancer screening or its confirmation and its prognosis 418-420.

Treatment solutions enables a single solution to protect clinical biological specimens and biomolecules, to inhibit endogenous DNAse, RNAse and proteinase, and to separate Large RNA/mRNA/ccfRNA from DNA/ccfDNA, Small RNA/miRNA/ccfmiRNA and protein. Treatment solutions also has following features and functions including differential solid phase binding of DNA/ccfDNA, large RNA/mRNA/ccfRNA, Small RNA/miRNA/ccfmiRNA and Carbohydrate on solid phase binding materials; preventing protein from precipitation in plasma or serum for effective isolation of ccfDNA, ccfRNA and ccfmiRNA without co-precipitation of protein when high concentration of ethanol is applied during isolation; removing inhibitors in plasma or serum from isolated ccfDNA, ccfRNA and ccfmiRNA; and purification of bio-molecules. Decontamination solution has the functions of decontamination of impurities and DNA from Large RNA/mRNA/ccfRNA, alteration of bio-molecules, protection and purification of bio-molecules by differential precipitation or differential solid phase binding to solid phase binding materials.

The present disclosure also relates to a kit for preparation of a specimen (such as a clinical biological specimen), isolation of a bio-molecule and analysis of a bio-molecule. The kit also comprises a step-wise methodology for the isolation of a specimen; reagents for protecting, lysing and inhibiting the specimen; a reagent for isolating a bio-molecule and a reagent for an assay for the isolated biomolecule. In some kits a reagent for protecting the specimen may also be present along with a reagent for doing a morphological assay on the protected specimen. The procedures for preparation of a specimen, protection, lysis and isolation of bio-molecules are well described in U.S. Pat. No. 8,530,228 (incorporated herein to its entirety). A kit, comprising: a core module, a decontaminant solution, a lysis solution, a plug-in module for the isolation of a bio-molecule from a biological specimen wherein the bio-molecule is analyzed to diagnosis a disease or a disease condition. The kit further comprises one or more plug-in modules for isolation of a number of bio-molecules from a biological specimen wherein the bio-molecule is analyzed to diagnosis a disease or a disease condition. Also, the kit may comprise of a staining kit to carry out morphological study wherein structural studies are done to diagnose a disease or a disease condition.

### EXAMPLES

### 1. Systems Preparation of Clinical Biological Specimens

Systems preparation of clinical biological specimens is the key to success for integrated and versatile methods for systems diagnosis of diseases with comprehensive understanding the cause, prevention, risk, seriousness, confirmation, treatment, triage, and prognosis of diseases. Systems preparation of clinical biological specimens integrates synchronously the methods of protection, isolation and alteration of specimens and bio-molecules for systems preparation of cells, tissues, and bio-molecules including DNA/ccfDNA, Large RNA/mRNA/ccfRNA, Small RNA/miRNA/ccfmiRNA, Protein, Lipid, Carbohydrates, and Metabolite simultaneously or individually from a variety of clinical biological specimens, which has been described with details in U.S. Pat. No. 8,530,228.

Methods of isolation, lysing clinical biological specimen, and further extracting, separating and purifying the distinct bio-molecule from lysate containing mixture of bio-molecules as seen in FIG. 3. For examples in brief, the Large RNA/mRNA/ccfRNA is isolated by centrifuging the tube with lysate at 13,000 rpm (16,000 g rcf) in micro-centrifuge or 16,000 g in a large centrifuge for 5 min at room temperature to separate Large RNA/mRNA/ccfRNA in pellet from DNA/ccfDNA and Small RNA/miRNA/ccfmiRNA, protein, lipid, carbohydrates, and metabolite in supernatant. The DNA/ccfDNA is isolated by centrifuging mixture of lasyte with 30-40% of ethanol at 13,000 rpm (16,000 g rcf) for 5 min at room temperature to pellet DNA/ccfDNA, or binding on affinity columns after isolation of Large RNA/mRNA/ccfRNA.

The Small RNA/miRNA/ccfmiRNA is isolated by centrifuging mixture of lysate with 50-60% of ethanol at 13,000 rpm (16,000 g rcf) for 5 min at room temperature to pellet Small RNA/miRNA/ccfmiRNA, or binding on affinity columns after isolation of DNA/ccfDNA. The Protein is isolated by centrifuging mixture of lysate with 90% of ethanol at 13,000 rpm (16,000 g rcf) for 5 min at room temperature to pellet protein. Lipids are isolated by adding 2.4 ml of Trimethylene bromochloride to about 800 ul of the saved supernatant of protein for isolation of lipids, carbohydrates and metabolites. The carbohydrate is isolated by add 80 ul of 2× Carb binder to about 80 ul of the saved aqueous phase for isolation of carbohydrates and metabolites after isolation of lipids, and bound on Con-A columns. The metabolites are isolated by collecting flow through from Con-A columns. Desalt from metabolites can be applied if necessary.

### 2. Systems Diagnosis of Cervical Cancer Through Systems Preparation of Cervical Secretion and Mucus

The integrated and versatile methods for systems diagnosis of disease has been implemented for cervical cancer through preparation and tests of cell, tissue and bio-molecules derived from cervical secretion and mucus containing cells. Since specimens, bio-molecules, test methods and mechanism of diseases are varied among different diseases, results and contents of the systems diagnosis of diseases are slightly different from general principle as shown in FIG. 1. The systems diagnosis of cervical cancer is achieved through comprehensive understanding the cause screen, treatment triage, pre-cancer screen, confirmation, and prognosis of cervical cancer as show in FIG. 4.

The materials for systems diagnosis of cervical cancer are derived from clinical biological specimens, including cells, tissue, and bio-molecules such as DNA, RNA, miRNA and protein. The cervical secretion and mucus containing cells is treated with a protecting, lysis and inhibiting solution selected from the group consisting of a variety of treatment solutions to protect histological structures and micromorphology of cells and tissues, and intactness of bio-molecules of the cervical secretion and mucus containing cells and/or to preserve the intactness of the bio-molecules in a lysate of the cervical secretion and mucus containing cells.

The protecting, lysis and inhibiting solution inhibits endogenous degrading enzymes in the cervical secretion and mucus. Protected cells and tissues are prepare for morphology based cytology and histology test. Protected bio-molecules including HPV DNA, E6E7 mRNA, miR-200a and miR-9 miRNA, and E6E7 protein are isolated for molecular tests, immunology, and mass spectrum test. HPV DNA, E6E7 mRNA, miR-200a and miR-9 miRNA, and E6E7 protein are isolated, after lysis or homogenization of cervical secretion and mucus, by performing a differential centrifugation process, a differential precipitation process and a differential solid phase binding process on the lysate without toxic phenol/chloroform extraction to separate the HPV DNA, E6E7 mRNA, miR-200a and miR-9 miRNA, and E6E7 protein.

The systems diagnosis of cervical cancer is carried out through tests of cells, tissues, and bio-molecules derived from cervical secretion and mucus on aspects of cytology and histological structures and micromorphology of epithelia cells and biopsy tissues; molecular biology test of HPV DNA, E6/E7 mRNA, miR-200a and miR-9; and immunology test of E6/E7 protein. Epithelia cells and biopsy tissues are tested by conventional cytology and histology routinely practice in pathology laboratory; HPV DNA is tested by methods of molecular test including Hybridization (HPV DNA Hybrid Capture assay from Qiagen Digene, U.S. Pat. No. 7,361,460), Polymerase Chain Reaction (PCR) (AMPLICOR HPV Test (CE-IVD) from Roche Diagnostics) or microarray; E6/E7 mRNA is tested by methods of molecular test, Reverse Transcription Polymerase Chain Reaction (RT-PCR), or APTIMA E6/E7 mRNA test from Hologic Gen-Probe (U.S. Pat. No. 7,354,719; incorporated herein in its entirety); E6/E7 protein is tested by method of immunoassay (U.S. Pat. No. 6,933,123); and miRNA 200a and miRNA-9 are tested by method of molecular test, RT-PCR (Cancer Research. 2010 Feb. 15; 70(4):1441-8.).

The results of above tests represent different aspects of the cervical cancer status. Molecular tests such as HPV DNA test and E6/E7 mRNA tests are primary screen methods with high sensitivity and efficiency for earlier stage screening. High risk HPV, such as HPV 16 or 18 have higher potent to cause cervical cancer, which should be taken seriously with aggressive actions; Expression of E6/E7 mRNA and protein indicates whether or not the HPV has become active in triggering the cancerous process. Detection of E6/E7 mRNA as a marker of high risk HPV infection will find out whether the infection may be dormant (harmless) or active (potentially harmful). Therefore, E6/E7 mRNA-based tests may help reduce the number of women who need to undergo further treatment, such as colposcopy.

Conventional cytology test is secondary screen method for pre-cancer. Cervical Intraepithelial Neoplasia (CIN) results can serve as further triage for primary screen tests. CIN1 is mild dysplasia and my not require colposcopy, whereas carcinoma In Situ (CIS)/CIN2/CIN3/are moderate and severe dysplasia and aggressive actions are advised. Conventional histology test will confirm the pathological diagnosis for adenocarcinoma in Situ, micro-invasive squamous carcinoma, invasive squamous or glandular carcinoma, which are indications for aggressive surgical, chemical or radiation treatments. miRNA 200a and miRNA-9 test serves prognosis of cervical cancer and predict patient survival. Both miR-200a and miR-9 could play important regulatory roles in cervical cancer control. In particular, miR-200a is likely to affect the metastatic potential of cervical cancer cells by coordinate suppression of multiple genes controlling cell motility.

The systems diagnosis of cervical cancer is achieved by integrating comprehensive parameters from preventive screen test of high risk HPV DNA to identify high risk of population to suffer from cervical cancer in the future; test of E6/E7 mRNA and E6/E7 protein for treatment triage; test of micromorphology changes of epithelia cells to screen population to have cervical pre-cancer; test of histology changes to confirm the diagnosis of cervical cancer; and test of expression signature miR-200a and miR-9 for cervical cancer prognosis. The results of systems diagnosis of cervical cancer will provide better management of cervical cancer than individualized and separated test results, increase the efficiency and reduce the cost for testing laboratory as well as patients, therefore benefit patients with cervical cancer.

The invention has been described using exemplary preferred embodiments. However, for those skilled in this field, the preferred embodiments can be easily adapted and modified to suit additional applications without departing from the spirit and scope of this invention. Thus, it is to be understood that the scope of the invention is not limited to the disclosed embodiments. On the contrary, it is intended to cover various modifications and similar arrangements based upon the same operating principle. The scope of the claims, therefore, should be accorded the broadest interpretations so as to encompass all such modifications and similar arrangements. Accordingly, the specification and drawings are to be regarded in an illustrative rather than a restrictive sense.

What is claimed is:

1. A method comprises:
(a) a biological sample;
(b) contacting a protecting, lysis and inhibiting solution to the biological specimen;
(c) forming a homogenate or a lysate;
(d) subjecting the homegenate/lysate to a differential centrifugation followed by a differential precipitation to form a sample;
(e) isolating a bio-molecule from the sample;
(f) assessing the bio-molecule; and
(g) analyzing the result for the diagnosis of a disease.

2. The method of claim 1, wherein a single sample is prepared from a biological specimen to isolate a bio-molecule.

3. The method of claim 1, wherein a single sample is prepared from a biological specimen to isolate a plurality of bio-molecules.

4. The method of claim 1, further comprise of performing morphological studies on the specimen contacted with the protecting solution.

5. The method of claim 1 wherein the specimen can be a solid specimen, a liquid specimen and/or a clinical biological specimen.

6. The method of claim 5, wherein the clinical biological specimen is a cervical cancer tissue.

7. The method of claim 1, wherein the homogenate/lysate comprise a large RNA/mRNA/ccfRNA, a DNA/ccfDNA, a small RNA/miRNA/ccfmiRNA and a protein.

8. The method of claim 1, wherein the bio-molecules include a DNA/ccfDNA, a large RNA/mRNA/ccfRNA, a small RNA/miRNA/ccfmiRNA, a protein, a lipid, a carbohydrate, and a metabolite.

9. The method of claim 1, wherein performing a differential centrifugation process and a differential precipitation process on the homogenate/lysate to separate the bio-molecule comprises the steps of:
(a) centrifuging the homogenate/lysate to separate and collect the large RNA/mRNA/ccfRNA as pellet 1 away from a supernatant 1 comprising DNA/ccfDNA, small RNA/miRNA/ccfmiRNA and protein; RNA/mRNA/ccfRNA, a small RNA/miRNA/ccfmiRNA, a protein, a lipid, a carbohydrate, and a metabolite.
(b) dissolving the pellet 1 comprising the large RNA/mRNA/ccfRNA in a decontamination solution comprising sodium iodide at 1-5 M, ammonium acetate at 1-10 M, sodium acetate at 0.1-5M, sodium citrate at 10-100 mM, sodium chloride (NaCl) at 0.1-5 M, and ethylene diamine tetraacetic acid dis odium salt dihydrate (EDTA) at 0.1-50 mM;
(c) periodically vortex the pellet 1 to dissolve the pellet thoroughly in the decontamination solution for five seconds at a time;
(d) centrifuging the dissolved pellet 1 of step (c) at 13000 rpm for 2 min at room temperature to remove insoluble contaminants as a new pellet and recover the large RNA/mRNA/ccfRNA in a supernatant 2;
(e) adding ethanol to the supernatant 2 to form a mixture comprising precipitated large RNA/mRNA/ccfRNA;
(f) centrifuging the mixture of step (e) to collect the precipitated large RNA/mRNA/ccfRNA as a pellet 2 and a supernatant 3 for discarding;
(g) washing the pellet 2 with a 75% ethanol solution;
(h) centrifuging to recollect pellet 2 as a pure sample of the large RNA/mRNA/ccfRNA after discard the supernatant 4;
(i) air drying of pellet 2; and
j) dissolving the air dried pellet 2 in RNAse and DNase free water.

10. The method of claim 9, further comprise extracting DNA/ccfDNA from the supernatant 1 by a differential centrifugation and a differential precipitation process without toxic phenol/chloroform extraction, wherein the extracting of DNA/ccfDNA comprises:
(a) adding ethanol to the supernatant 1 to precipitate the DNA/ccfDNA;
(b) collecting the precipitated DNA/ccfDNA by centrifugation at 13000 rpm as a pellet 3 comprising the DNA/ccfDNA and a supernatant 5 comprising small RNA/miRNA/ccfmiRNA and protein;
(c) washing the pellet 3 with a 75% ethanol solution; and
(d) centrifuging and collecting the pure sample of the DNA/ccfDNA.

11. A method comprises:
(a) a biological sample;
(b) contacting a protecting, lysis and inhibiting solution to the biological specimen;
(c) forming a homogenate or a lysate;
(d) subjecting the homegenate/lysate to a differential solid phase binding followed by a differential precipitation to form a sample;
(e) isolating a bio-molecule from the sample;
(f) assessing the bio-molecule; and
(g) analyzing the result for the diagnosis of a disease.

12. The method of claim 11, wherein performing a differential solid phase binding on the homogenate/lysate to separate the bio-molecule comprises the steps of:
(a) centrifuging the homogenate/lysate to separate and collect the large RNA/mRNA/ccfRNA as pellet 1 away from a supernatant 1 comprising DNA/ccfDNA, small RNA/miRNA/ccfmiRNA and protein;
(b) dissolving the pellet 1 comprising the large RNA/mRNA/ccfRNA in a decontamination solution comprising sodium iodide at 1-5 M, ammonium acetate at 1-10 M, sodium acetate at 0.1-5M, sodium citrate at 10-100 mM, sodium chloride (NaCl) at 0.1-5 M, and ethylene diamine tetraacetic acid dis odium salt dihydrate (EDTA) at 0.1-50 mM;
(c) periodically vortex the pellet 1 to dissolve the pellet thoroughly in the decontamination solution for five seconds at a time;
(d) centrifuging the dissolved pellet 1 of step (c) at 13000 rpm for 2 min at room temperature to remove insoluble contaminants as a new pellet and recover the large RNA/mRNA/ccfRNA in a supernatant 2;
(e) adding ethanol and solid phase binding materials to the supernatant 2 to bind large RNA/mRNA/ccfRNA on solid phase binding materials to form bond solid phase binding materials 1;
(f) Separating the bond solid phase binding materials of step (e) to collect the bond large RNA/mRNA/ccfRNA on solid phase binding materials as the bond large RNA/mRNA/ccfRNA and a supernatant 3 for discarding;
(g) washing the bond large RNA/mRNA/ccfRNA with a 75% ethanol solution;
(h) Separating to recollect the bond large RNA/mRNA/ccfRNA as a pure sample of the large RNA/mRNA/ccfRNA after discard the supernatant 4;
(i) air drying of the bond large RNA/mRNA/ccfRNA; and
(j) eluting the air dried bond large RNA/mRNA/ccfRNA in RNAse and DNase free water.

13. The method of claim 12, further comprise extracting DNA/ccfDNA, small RNA/miRNA/ccfmiRNA, and protein from the supernatant 1 by a differential solid phase binding without toxic phenol/chloroform extraction, wherein the extracting of DNA/ccfDNA, small RNA/miRNA/ccfmiRNA, and protein comprises:
(a) adding ethanol and solid phase binding materials to the supernatant 1 to bind the DNA/ccfDNA to form bond solid phase binding materials 2;
(b) Separating the bond solid phase binding materials 2 of step (a) to collect the bond DNA/ccfDNA on solid phase binding materials 2 as the bond DNA/ccfDNA and a supernatant 5 comprising small RNA/miRNA/ccfmiRNA and protein;
(c) washing the bond DNA/ccfDNA with a 75% ethanol solution; and
(d) eluting the air dried bond DNA/ccfDNA and collecting the pure sample of the DNA/ccfDNA.
(e) adding ethanol and solid phase binding materials to the supernatant 5 to bind the small RNA/miRNA/ccfmiRNA to form bond solid phase binding materials 3;
(f) Separating the bond solid phase binding materials 3 of step (e) to collect the bond small RNA/miRNA/ccfmiRNA on solid phase binding materials 3 as the bond small RNA/miRNA/ccfmiRNA and a supernatant 6 comprising protein;
(g) washing the small RNA/miRNA/ccfmiRNA with a 75% ethanol solution; and
(h) eluting the air dried bond small RNA/miRNA/ccfmiRNA and collecting the pure sample of the small RNA/miRNA/ccfmiRNA.
(i) adding ethanol to the supernatant 6 to form a mixture comprising precipitated protein;
(j) centrifuging the mixture of step (i) to collect the precipitated protein as a pellet 3 and a supernatant 7 containing lipid, carbohydrates, and other metabolites;
(k) washing the pellet 3 with a 75% ethanol solution;
(l) centrifuging to recollect pellet 3 as a pure sample of the protein after collecting the supernatant 8 for further extraction of lipid, carbohydrates, and other metabolites;
(m) air drying of pellet 3; and
(n) dissolving the air dried pellet 3 as protein.

14. The method of claim 11, wherein the solid phase binding is done with a magnetic particle coated with a material which bind to a nucleic acid.

15. The method of claim 14, wherein the material is a glass fiber.

16. The method of claim 11, wherein a single sample is prepared from a biological specimen to isolate a plurality of bio-molecules.

17. The method of claim 11, wherein the bio-molecules include a DNA/ccfDNA, a large RNA/mRNA/ccfRNA, a small RNA/miRNA/ccfmiRNA, a protein, a lipid, a carbohydrate, and a metabolite.

18. A kit, comprising: a core module, a decontaminant solution, a lysis solution, a plug-in module for the isolation of a bio-molecule from a biological specimen wherein the bio-molecule is analyzed to diagnosis a disease or a disease condition.

19. The kit of claim 18, further comprising: one or more plug-in modules for isolation of a number of bio-molecules from a biological specimen wherein the bio-molecule is analyzed to diagnosis a disease or a disease condition.

20. The kit of claim 19, further comprising: a staining kit to carry out morphological study wherein structural studies are done to diagnose a disease or a disease condition.
